(19) United States
(12) Patent Application Publication
(10) Pub. No.: US 2010/0190217 A1
(43) Pub. Date: Jul. 29, 2010

(54) METHOD FOR PRODUCING L-LYSINE

(76) Inventors: HIDETAKA DOI, Kawasaki-shi (JP); TAKUJI UEDA, Kawasaki-shi (JP)

Correspondence Address: CERMAK NAKAJIMA LLP;ACS LLC, 127 S. Peyton Street, Suite 210, ALEXANDRIA, VA, 22314, US

(21) Appl. No.: 12/691,211

(22) Filed: Jan. 21, 2010

Related U.S. Application Data
(63) Continuation of application No. PCT/JP2008/063126, filed on Jul. 22, 2008.

(30) Foreign Application Priority Data
Jul. 23, 2007 (JP) 2007-190795

(51) Int. Cl. C12P 13/08; C12N 1/21

(52) U.S. Cl. 435/115; 435/252.33

(57) ABSTRACT

L-Lysine is produced by culturing in a medium an Escherichia coli having an L-lysine-producing ability, which has been modified to decrease the activity or activities of one or more enzymes of the meso-α,ε-diaminopimelic acid synthesis pathway, for example, 2,3,4,5-tetrahydropyridine-2,6-dicarboxylate N-succinyltransferase, succinyldiaminopimelate transaminase, succinyldiaminopimelate desuccinylase, and diaminopimelate epimerase, and into which a gene coding for diaminopimelate dehydrogenase has been introduced, and collecting L-lysine from the medium.

This application is a continuation under 35 U.S.C. §120 of PCT Patent Application No. PCT/JP2008/063126, filed Jul. 22, 2008, which claims priority under 35 U.S.C. §119 to Japanese Patent Application No. 2007-190795, filed on Jul. 23, 2007, which are incorporated in their entireties by reference. The Sequence Listing in electronic format filed herewith is also hereby incorporated by reference in its entirety (File Name: US-420_Seq_List; File Size: 90 KB; Date Created Jan. 21, 2010).

### BACKGROUND OF THE INVENTION

1. Technical Field

The present invention relates to a method for producing L-lysine utilizing Escherichia coli. L-Lysine is an essential amino acid, and is often utilized as a component in drug formulations and various nutritional mixtures. L-Lysine is also used as an animal feed additive.

2. Background Art

L-Amino acids such as L-lysine are industrially produced by fermentation using bacteria such as coryneform and Escherichia bacteria that are able to produce such L-amino acids. These bacterial strains can be isolated from nature, or artificial mutant strains can be created, including recombinant strains which have enhanced activity of L-amino acid biosynthesis enzymes via gene recombination, and so forth. L-amino acid production can be improved in these mutant strains. Examples of methods for producing L-lysine include the methods described in Japanese Patent Laid-open (KOKAI) Nos. 10-165180, 11-192088, 2000-253879, and 2001-057896.

Besides increasing expression of an enzyme of a biosynthetic pathway characteristic to the target amino acid, other methods for increasing production of L-amino acids such as L-lysine have been developed, such as by modifying the respiratory chain pathway to improve energy efficiency (Japanese Patent Laid-open No. 2002-17363), and amplifying a nicotinamide nucleotide transhydrogenase gene to increase nicotinamide adenine dinucleotide phosphate-producing ability (Japanese Patent No. 2817400).

Moreover, bacteria with modifications to a pathway common to biosyntheses of various amino acids include bacteria in which the anaplerotic pathway is modified, such as the L-lysine-producing coryneform bacterium in which pyruvate carboxylase activity is increased (Japanese Patent Laid-open based on PCT application in foreign language (KOHYO) No. 2002-508921), the pyruvate kinase-deficient L-lysine-producing Escherichia bacterium (International Patent Publication WO03/008600), and the L-lysine-producing coryneform bacterium which is deficient in maleate quinone oxidoreductase (U.S. Patent Published Application No. 2003/0044943).

Meso-α,ε-diaminopimelic acid (also referred to as “meso-DAP”) is known to be a precursor to L-lysine. Meso-DAP is also indispensable for growth of bacteria since it is a constituent component of cell walls. Meso-DAP is known to be synthesized from a precursor, 2,3,4,5-tetrahydropyridine-2,6-dicarboxylic acid (henceforth also referred to as “THDP”), with the help of four enzymes, 2,3,4,5-tetrahydropyridine-2,6-dicarboxylate N-succinyltransferase (also referred to as “DapD”, Richaud, C. et al., J. Biol. Chem., 259 (23):14824-14828, 1984), succinyldiaminopimelate transaminase (also referred to as “DapC”, Heimberg, H. et al., Gene, 90(1):69-78, 1990), succinyldiaminopimelate desuccinylase (also referred to as “DapE”, Bouvier, J. et al., J. Bacteriol., 174 (16):5265-71, 1992), and diaminopimelate epimerase (also referred to as “DapF”, Wiseman, J. S. et al., J. Biol. Chem., 259 (14):8907-14, 1984). All of these enzymes belong to the meso-DAP synthesis pathway. It has been reported that coryneform bacteria have a different meso-DAP synthesis pathway which utilizes THDP as a precursor, and meso-DAP is synthesized from THDP in one step using meso-DAP dehydrogenase (also referred to as “diaminopimelate dehydrogenase” or “DDH”). Expression of DDH is known to be useful for the production of meso-DAP (Japanese Patent Laid-open No. 61-289887). Moreover, during the investigation of the rate-limiting step of L-lysine biosynthesis in Escherichia bacteria, it was found that the gene coding for DDH from coryneform bacterium, when introduced in the bacteria, can be used to increase L-lysine production, instead of increasing an activity of a meso-DAP synthesis pathway enzyme (U.S. Pat. No. 6,040,160). However, it has not been previously reported that when DDH is expressed in an Escherichia bacterium, decreasing the activity of an enzyme belonging to the meso-DAP synthesis pathway can be effective for increasing L-lysine production.

### SUMMARY OF THE INVENTION

An aspect of the present invention is to provide an Escherichia coli which has improved L-lysine-producing ability, and a method for producing L-lysine utilizing such an Escherichia coli.

It has been found that the L-lysine-producing ability of Escherichia coli can be improved by modifying an Escherichia coli so that the activity of an enzyme belonging to the meso-DAP synthetic pathway is decreased, in combination with introducing a gene coding for diaminopimelate dehydrogenase.

It is an aspect of the present invention to provide an Escherichia coli bacterium which is able to produce L-lysine, wherein said bacterium has been modified to decrease an activity or activities of one or more enzymes of the meso-α,ε-diaminopimelic acid synthesis pathway, and wherein a gene coding for diaminopimelate dehydrogenase has been introduced into said bacterium.

It is a further aspect of the present invention to provide the aforementioned Escherichia coli bacterium, wherein the enzymes of the meso-α,ε-diaminopimelic acid synthesis pathway are selected from the group consisting of 2,3,4,5-tetrahydropyridine-2,6-dicarboxylate N-succinyltransferase, succinyldiaminopimelate transaminase, succinyldiaminopimelate desuccinylase, and diaminopimelate epimerase.

It is a further aspect of the present invention to provide the aforementioned Escherichia coli bacterium, wherein the 2,3,4,5-tetrahydropyridine-2,6-dicarboxylate N-succinyltransferase, succinyldiaminopimelate transaminase, succinyldiaminopimelate desuccinylase, and diaminopimelate epimerase are encoded by the dapD gene, dapC gene, dapE gene, and dapF gene, respectively.

It is a further aspect of the present invention to provide the aforementioned Escherichia coli bacterium, wherein the activity or activities are decreased by a method selected from the group consisting of 1) decreasing expression of the gene or genes, 2) disrupting the gene or genes, and 3) combinations thereof.

It is a further aspect of the present invention to provide the aforementioned Escherichia coli bacterium, which has been modified to decrease at least the activity of 2,3,4,5-tetrahydropyridine-2,6-dicarboxylate N-succinyltransferase.

It is a further aspect of the present invention to provide the aforementioned Escherichia coli bacterium, wherein the gene coding for diaminopimelate dehydrogenase is the ddh gene of a coryneform bacterium.

It is a further aspect of the present invention to provide the aforementioned Escherichia coli bacterium, wherein the 2,3,4,5-tetrahydropyridine-2,6-dicarboxylate N-succinyltransferase is selected from the group consisting of:

(a) the protein comprising the amino acid sequence of SEQ ID NO: 2, and

(b) the protein comprising the amino acid sequence of SEQ ID NO: 2, but wherein one or several amino acid residues are substituted, deleted, inserted or added, and the protein has 2,3,4,5-tetrahydropyridine-2,6-dicarboxylate N-succinyltransferase activity.

It is a further aspect of the present invention to provide the aforementioned Escherichia coli bacterium, wherein the succinyldiaminopimelate transaminase is selected from the group consisting of:

(a) the protein comprising the amino acid sequence of SEQ ID NO: 4, and

(b) the protein comprising the amino acid sequence of SEQ ID NO: 4, but wherein one or several amino acid residues are substituted, deleted, inserted or added, and the protein has succinyldiaminopimelate transaminase activity.

It is a further aspect of the present invention to provide the aforementioned Escherichia coli bacterium, wherein the succinyldiaminopimelate desuccinylase is selected from the group consisting of:

(a) the protein comprising the amino acid sequence of SEQ ID NO: 6, and

(b) the protein comprising the amino acid sequence of SEQ ID NO: 6, but wherein one or several amino acid residues are substituted, deleted, inserted or added, and the protein has succinyldiaminopimelate desuccinylase activity.

It is a further aspect of the present invention to provide the aforementioned Escherichia coli bacterium, wherein the diaminopimelate epimerase is selected from the group consisting of:

(a) the protein comprising the amino acid sequence of SEQ ID NO: 8, and

(b) the protein comprising the amino acid sequence of SEQ ID NO: 8, but wherein one or several amino acid residues are substituted, deleted, inserted or added, and the protein has diaminopimelate epimerase activity.

It is a further aspect of the present invention to provide the aforementioned Escherichia coli bacterium, wherein the dapD gene is selected from the group consisting of:

(a) a DNA comprising the nucleotide sequence of SEQ ID NO: 1, and

(b) a DNA which is able to hybridize with the nucleotide sequence of SEQ ID NO: 1, or a probe which can be prepared from the nucleotide sequence, under stringent conditions, and wherein said DNA encodes a protein having 2,3,4,5-tetrahydropyridine-2,6-dicarboxylate N-succinyltransferase activity.

It is a further aspect of the present invention to provide the aforementioned Escherichia coli bacterium, wherein the dapC gene is selected from the group consisting of:

(a) the DNA comprising the nucleotide sequence of SEQ ID NO: 3, and

(b) a DNA which is able to hybride with the nucleotide sequence of SEQ ID NO: 3, or a probe which can be prepared from the nucleotide sequence, under stringent conditions, and wherein said DNA encodes a protein having succinyldiaminopimelate transaminase activity.

It is a further aspect of the present invention to provide the aforementioned Escherichia coli bacterium, wherein the dapE gene is selected from the group consisting of:

(a) the DNA comprising the nucleotide sequence of SEQ ID NO: 5, and

(b) a DNA which is able to hybridize with the nucleotide sequence of SEQ ID NO: 5, or a probe which can be prepared from the nucleotide sequence, under stringent conditions, and wherein said DNA encodes a protein having succinyldiaminopimelate desuccinylase activity.

It is a further aspect of the present invention to provide the aforementioned Escherichia coli bacterium, wherein the dapF gene is selected from the group consisting of:

(a) the DNA comprising the nucleotide sequence of SEQ ID NO: 7, and

(b) a DNA which is able to hybridize with the nucleotide sequence of SEQ ID NO: 7, or a probe which can be prepared from the nucleotide sequence, under stringent conditions, and wherein said DNA encodes a protein having diaminopimelate epimerase activity.

It is a further aspect of the present invention to provide the aforementioned Escherichia coli bacterium, wherein the diaminopimelate dehydrogenase is selected from the group consisting of:

(a) the protein comprising the amino acid sequence selected from the group consisting of SEQ ID NO: 10, 12, 14, 16, and 18,

(b) the protein comprising the amino acid sequence selected from the group consisting of SEQ ID NO: 10, 12, 14, 16, and 18, but wherein one or several amino acid residues are substituted, deleted, inserted or added in said amino acid sequence, and the protein has diaminopimelate dehydrogenase activity.

It is a further aspect of the present invention to provide the aforementioned Escherichia coli bacterium, wherein the ddh gene is selected from the group consisting of:

(a) the DNA comprising the nucleotide sequence selected from the group consisting of SEQ ID NO: 9, 11, 13, 15, and 17, and

(b) a DNA which is able to hybridize with the nucleotide sequence selected from the group consisting of SEQ ID NO: 9, 11, 13, 15, and 17 or a probe, which can be prepared from the nucleotide sequence, under stringent conditions, and wherein said DNA encodes a protein having diaminopimelate dehydrogenase activity.

It is a further aspect of the present invention to provide the aforementioned Escherichia coli bacterium, which further has: a) a dihydrodipicolinate synthase which is desensitized to feedback inhibition by L-lysine, b) an aspartokinase which is desensitized to feedback inhibition by L-lysine, and c) enhanced activity of dihydrodipicolinate reductase.

It is a further aspect of the present invention to provide a method for producing L-lysine, which comprises culturing the aforementioned Escherichia coli bacterium in a medium and collecting L-lysine from the medium.

### DETAILED DESCRIPTION OF THE PREFERRED EMBODIMENTS

Hereinafter, the present invention will be explained in detail.

<1>Escherichia coli

The Escherichia coli bacterium in accordance with the presently disclosed subject matter is able to produce L-lysine, and has been modified to decrease the activity or activities of one or more enzymes of the meso-α,ε-diaminopimelic acid synthesis pathway. The gene coding for diaminopimelate dehydrogenase has also been introduced into this Escherichia coli.

In addition, this bacterium can have a dihydrodipicolinate synthase which is desensitized to feedback inhibition by L-lysine, an aspartokinase which is desensitized to feedback inhibition by L-lysine, and enhanced activity of dihydrodipicolinate reductase.

The bacterium can be obtained by modifying a parent or wild-type strain of Escherichia coli that is able to produce L-lysine so that an activity of an enzyme belonging to the meso-DAP synthesis pathway is decreased, and then further introducing a gene coding for diaminopimelate dehydrogenase. The order of the modification for decreasing an activity of an enzyme belonging to the meso-DAP synthesis pathway, and the introduction of a gene coding for diaminopimelate dehydrogenase, is not particularly limited. Moreover, the L-lysine-producing ability can be imparted between or after the modification and the gene introduction.

The parent or wild-type strain of Escherichia coli which can be used to obtain the bacterium is not particularly limited. However, specific examples include, for example, those described in the work of Neidhardt et al. (Neidhardt F. C. et al., Escherichia coli and Salmonella typhimurium, American Society for Microbiology, Washington D.C., 1029, table 1). Specific examples include the Escherichia coli W3110 (ATCC 27325), Escherichia coli MG1655 (ATCC 47076), and so forth, which are derived from the prototype wild-type strain, K12 strain.

These strains are available from, for example, American Type Culture Collection (Address: 12301 Parklawn Drive, Rockville, Md. 20852, United States of America). That is, registration numbers are given to each of the strains, and the strains can be ordered using these registration numbers. The registration numbers of the strains are listed in the catalogue of the American Type Culture Collection.

<1-1> Impartation of L-Lysine-Producing Ability and Escherichia coli Bacteria Having L-Lysine-Producing Ability

Examples of L-lysine-producing Escherichia coli bacteria include mutants that are resistant to an L-lysine analogue. L-Lysine analogues inhibit growth of Escherichia coli, but this inhibition is fully or partially desensitized when L-lysine is present in the medium. Examples of the L-lysine analogues include, but are not limited to, oxalysine, lysine hydroxamate, S-(2-aminoethyl)-L-cysteine (AEC), γ-methyllysine, α-chlorocaprolactam, and so forth. Mutants that are resistant to these lysine analogues can be obtained by subjecting Escherichia coli bacteria to a conventional artificial mutagenesis. Specific examples of bacterial strains that are able to produce L-lysine include Escherichia coli AJ11442 (FERM BP-1543, NRRL B-12185; see U.S. Pat. No. 4,346,170) and Escherichia coli VL611. In these microorganisms, aspartokinase is desensitized to feedback inhibition by L-lysine.

The WC196 strain can be used as an L-lysine-producing bacterium of Escherichia coli. This bacterial strain was bred by conferring AEC resistance to the W3110 strain, which was derived from Escherichia coli K-12. The resulting strain was designated Escherichia coli AJ13069 and was deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Dec. 6, 1994 and assigned an accession number of FERM P-14690. Then, the deposit was converted to an international deposit under the provisions of the Budapest Treaty on Sep. 29, 1995, and assigned an accession number of FERM BP-5252 (U.S. Pat. No. 5,827,698).

Examples of parent strains that can be used to derive L-lysine-producing bacteria also include strains in which expression of one or more genes encoding an L-lysine biosynthetic enzyme is increased. Examples of such genes include, but are not limited to, genes coding for dihydrodipicolinate synthase (dapA), aspartokinase (lysC), dihydrodipicolinate reductase (dapB), diaminopimelate decarboxylase (lysA), phosphoenolpyrvate carboxylase (ppc), aspartate semialdehyde dehydrogenease (asd), and aspartase (aspA) (EP 1253195 A). In addition, parental strains can have an increased level of expression of the gene involved in energy efficiency (cyo) (EP 1170376 A), the gene coding for nicotinamide nucleotide transhydrogenase (pntAB) (U.S. Pat. No. 5,830,716), the ybjE gene (WO2005/073390), the gene coding for glutamate dehydrogenase (gdhA, Gene, 23:199-209 (1983)), or a combination thereof. Abbreviations for the genes of the enzymes are shown in the parentheses.

The nucleotide sequence of the lysC gene of Escherichia coli is shown in SEQ ID NO: 21, and the encoded amino acid sequence of aspartokinase is shown in SEQ ID NO: 22. The nucleotide sequence of the dapA gene of Escherichia coli is shown in SEQ ID NO: 23, and the encoded amino acid sequence of dihydrodipicolinate synthase is shown in SEQ ID NO: 24. Furthermore, the nucleotide sequence of the dapB gene of Escherichia coli is shown in SEQ ID NO: 25, and the encoded amino acid sequence of dihydrodipicolinate reductase is shown in SEQ ID NO: 26.

It is known that the wild-type dihydrodipicolinate synthase derived from Escherichia coli is subject to feedback inhibition by L-lysine, and it is known that the wild-type aspartokinase derived from Escherichia coli is subject to suppression and feedback inhibition by L-lysine. Therefore, when the dapA and lysC genes are used, dapA and lysC genes coding for mutant enzymes that are desensitized to the feedback inhibition by L-lysine can be used.

An example of DNA encoding a mutant dihydrodipicolinate synthetase which is desensitized to feedback inhibition by L-lysine include a DNA encoding a protein which has the amino acid sequence of SEQ ID NO: 24, but in which the histidine residue at position 118 is replaced by tyrosine residue. An example of DNA encoding a mutant aspartokinase which is desensitized to feedback inhibition by L-lysine include a DNA encoding an AKIII having the amino acid sequence of SEQ ID NO: 22 in which the threonine residue at position 352, the glycine residue at position 323, and the methionine residue at position 318 are replaced by isoleucine, asparagine and isoleucine residues, respectively (U.S. Pat. No. 5,661,012 and U.S. Pat. No. 6,040,160). Such mutant DNAs can be obtained by site-specific mutagenesis using PCR or the like.

Wide host-range plasmids RSFD80, pCAB1, and pCABD2 derived from RSF1010 are known as plasmids containing a mutant dapA gene encoding a mutant Escherichia coli dihydrodipicolinate synthase and a mutant lysC gene encoding a mutant Escherichia coli aspartokinase (U.S. Pat. No. 6,040,160). The Escherichia coli JM109 strain transformed with RSFD80 was named AJ12396 (U.S. Pat. No. 6,040,160), and the strain was deposited at the National Institute of Bioscience and Human-Technology, Agency of Industrial Science and Technology, Ministry of International Trade and Industry (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Oct. 28, 1993 and assigned an accession number of FERM P-13936, and the deposit was then converted to an international deposit under the provisions of Budapest Treaty on Nov. 1, 1994 and assigned an accession number of FERM BP-4859. RSFD80 can be obtained from the AJ12396 strain by a conventional method. pCAB1 was prepared by further inserting the dapB gene of Escherichia coli into RSFD80 described above. Furthermore, pCABD2 was prepared by further inserting the ddh gene of Brevibacterium lactofermentum (Corynebacterium glutamicum) 2256 strain (ATCC 13869) into pCAB1 described above (U.S. Pat. No. 6,040,160).

Examples of L-lysine-producing bacteria or parent strains which can be used to derive such bacteria also include strains in which the activity of an enzyme that catalyzes a reaction which branches off from the L-lysine biosynthesis pathway and produces a compound other than L-lysine is decreased or made deficient. Examples of such enzymes include homoserine dehydrogenase, lysine decarboxylase (U.S. Pat. No. 5,827,698), and malic enzyme (WO2005/010175). Expression of both the cadA and ldcC genes encoding lysine decarboxylase can be decreased in order to decrease or delete the lysine decarboxylase activity (WO2006/038695).

In the bacterium in accordance with the presently disclosed subject matter, in order to enhance glycerol assimilation, expression of the glpR gene (EP 1715056) can be attenuated, or expression of glycerol metabolism genes (EP 1715055 A), such as glpA, glpB, glpC, glpD, glpE, glpF, glpG, glpK, glpQ, glpT, glpX, tpiA, gldA, dhaK, dhaL, dhaM, dhaR, fsa and talC, can be enhanced.

<1-2> Construction of the Escherichia coli

Hereinafter, the modification for reducing the activity of an enzyme belonging to the meso-DAP synthesis pathway, and the introduction of a gene coding for diaminopimelate dehydrogenase, will be explained.

The meso-DAP synthesis pathway of Escherichia coli generates meso-DAP (meso-2,6-diaminopimalate, meso-α,ε-diaminopimelate, or meso-diaminoheptanedioate) from (S)-2,3,4,5-tetrahydropyridine-2,6-dicarboxylate ((S)-2,3,4,5-tetrahydrodipicolinate), and is catalyzed by the enzymes for the following four reactions which occur step-wise. These reactions are reversible. In Escherichia coli, the meso-DAP synthesis pathway is also called the DapDCEF pathway.

### 1) DapD (2,3,4,5-tetrahydropyridine-2,6-dicarboxylate N-succinyltransferase, EC 2.3.1.117)

Succinyl-CoA+(S)-2,3,4,5-tetrahydropyridine-2,6-dicarboxylate+H₂O->CoA+N-succinyl-L-2-amino-6-oxoheptanedioate

DapD is encoded by the dapD gene. The sequence of the dapD gene of Escherichia coli is shown in SEQ ID NO: 1, and the amino acid sequence of DapD is shown in SEQ ID NO: 2.

The enzymatic activity of DapD can be measured by referring to the method of S. A, Simms et al. (J. Biol. Chem., 1984, March 10; 259(5):2734-2741).

### 2) DapC (succinyldiaminopimelate transaminase, also referred to as SDAP aminotransferase, EC 2.6.1.17)

N-Succinyl-LL-2,6-diaminoheptanedioate+2-oxoglutarate->N-succinyl-L-2-amino-6-oxoheptanedioate+L-glutamate

DapC is encoded by the dapC gene. The sequence of the dapC gene of Escherichia coli is shown in SEQ ID NO: 3, and the amino acid sequence is shown in SEQ ID NO: 4.

The enzymatic activity of DapC can be measured by the method of Thilo, M. et al. (J. Bacteriol., 2000, July; 182 (13):3626-3631).

### 3) DapE (succinyldiaminopimelate desuccinylase, also referred to as SDAP desuccinylation enzyme, EC 3.5.1.18)

N-Succinyl-LL-2,6-diaminoheptanedioate+H₂O->succinate+LL-2,6-diaminoheptanedioate

DapE is encoded by the dapE gene. The sequence of the dapE gene of Escherichia coli is shown in SEQ ID NO: 5, and the amino acid sequence is shown in SEQ ID NO: 6.

The enzymatic activity of DapE can be measured by the method of Lin, Y. K. et al. (J. Biol. Chem., 1988, February 5; 263(4):1622-1627).

### 4) DapF (diaminopimelate epimerase, EC 5.1.1.7)

LL-2,6-Diaminoheptanedioate->meso-2,6-diaminopimalate

DapF is encoded by the dapF gene. The sequence of the dapF gene of Escherichia coli is shown in SEQ ID NO: 7, and the amino acid sequence is shown in SEQ ID NO: 8.

The enzymatic activity of DapF can be measured by referring to the method of Wiseman, J. S. et al. (J. Biol. Chem., 1984, July 25; 259(14):8907-8914).

Furthermore, DDH (diaminopimelate dehydrogenase, EC 1.4.1.16) acts to reversibly generate (S)-2,3,4,5-tetrahydropyridine-2,6-dicarboxylate from meso-2,6-diaminopimalate, and catalyzes the following reaction.

Meso-2,6-diaminopimalate+H₂O+NADP⁺->(S)-2,3,4,5-tetrahydropyridine-2,6-dicarboxylate+NH₃+NADPH+H⁺

The enzymatic activity of DDH can be measured by referring to the method of Misono, H. et al. (J. Biol. Chem., 255, 10599-10605, 1980).

Although the ddh gene is not native to Escherichia bacteria, and so these Escherichia do not have the ddh gene, a ddh gene of a coryneform bacterium such as a ddh gene from Corynebacterium glutamicum (SEQ ID NO: 9), Brevibacterium lactofermentum (SEQ ID NO: 11), and Corynebacterium efficiens (SEQ ID NO: 13) can be used.

The ddh gene of Corynebacterium glutamicum ATCC 13032 (NCg12528) is registered as Genbank NP_{—}601818.2. GI:23308957, and the ddh gene of Corynebacterium efficiens (CE2498) is registered as NP_{—}739108.1. GI:25029054.

Besides the ddh gene of coryneform bacteria, the ddh gene of Herminiimonas arsenicoxydans (SEQ ID NO: 15), and the ddh gene of Bacteroides thetaiotaomicron (SEQ ID NO: 17) can be used. The ddh gene of Herminiimonas arsenicoxydans is registered as Genbank YP_{—}001100730.1 GI:134095655, and the ddh gene of Bacteroides thetaiotaomicron is registered as NP 810892.1. GI:29347389.

The aforementioned genes and the aforementioned L-lysine biosynthesis enzyme genes are not limited to the gene information described above or to genes having known sequences, but also include genes having conservative mutations such as homologues of the above native genes and artificially modified genes, so long as functions of the encoded proteins are not impaired. That is, a gene coding for an amino acid sequence of a known protein, but which includes substitutions, deletions, insertions, additions or the like of one or several amino acid residues at one or several positions can be used.

Although number meant by the phrase “one or several” can differ depending on the position in the three-dimensional structure of the protein or the type of amino acid residue being changed, specifically, it can be 1 to 20, 1 to 10 in another example, 1 to 5 in another example. The conservative mutation is typically a conservative substitution. The conservative substitution can take place mutually among Phe, Trp and Tyr, if the substitution site is an aromatic amino acid; among Leu, Ile and Val, if the substitution site is a hydrophobic amino acid; between Gln and Asn, if it is a polar amino acid; among Lys, Arg and His, if it is a basic amino acid; between Asp and Glu, if it is an acidic amino acid; and between Ser and Thr, if it is an amino acid having hydroxyl group. Specific examples of substitutions considered to be conservative substitutions include: substitution of Ser or Thr for Ala; substitution of Gln, His or Lys for Arg; substitution of Glu, Gln, Lys, His or Asp for Asn; substitution of Asn, Glu or Gln for Asp; substitution of Ser or Ala for Cys; substitution of Asn, Glu, Lys, His, Asp or Arg for Gln; substitution of Gly, Asn, Gln, Lys or Asp for Glu; substitution of Pro for Gly; substitution of Asn, Lys, Gln, Arg or Tyr for His; substitution of Leu, Met, Val or Phe for Ile; substitution of Ile, Met, Val or Phe for Leu; substitution of Asn, Glu, Gln, His or Arg for Lys; substitution of Ile, Leu, Val or Phe for Met; substitution of Trp, Tyr, Met, Ile or Leu for Phe; substitution of Thr or Ala for Ser; substitution of Ser or Ala for Thr; substitution of Phe or Tyr for Trp; substitution of His, Phe or Trp for Tyr; and substitution of Met, Ile or Leu for Val. These substitutions, deletions, insertions, additions, inversions or the like of amino acid residues as described above can also include a naturally occurring mutation based on individual differences, differences in species of microorganisms from which the genes are derived (mutant or variant), and so forth. Such a gene can be obtained by modifying a nucleotide sequence of a known gene by, for example, site-specific mutagenesis, so that substitution, deletion, insertion or addition of an amino acid residue or residues occurs at a specific site in the encoded protein.

Furthermore, a gene having the aforementioned conservative mutation can be a gene coding for a protein showing a homology of 80% or more, 90% or more in another example, 95% or more in another example, 97% or more in another example, to the entire sequence of the encoded protein, and having a function equivalent to that of the corresponding wild-type protein. In this specification, the term “homology” can also refer to “identity”.

Furthermore, codons of the gene sequences can be replaced with codons which are easily used by the host into which the genes are introduced.

Genes having a conservative mutation can also be obtained by a method typically used for mutagenesis, such as by treatment with a mutagen.

Moreover, the genes can also be able to hybridize with a probe that can be prepared from the known gene sequence, for example, the aforementioned gene sequences and sequences complementary to them, under stringent conditions, and the gene can encode for a protein having a function equivalent to that of the corresponding known gene product. The “stringent conditions” include when a so-called specific hybrid is formed, and non-specific hybrid is not formed. Examples of the stringent conditions include, for example, conditions under which DNAs showing high homology to each other, for example, DNAs having a homology of, for example, not less than 80%, not less than 90% in another example, not less than 95% in another example, not less than 97% in another example, hybridize with each other, and DNAs having homology lower than the above level do not hybridize with each other, and conditions of washing in ordinary Southern hybridization, i.e., conditions of washing once, twice or three times in another example, at salt concentrations and temperature of 1×SSC, 0.1% SDS at 60° C., 0.1×SSC, 0.1% SDS at 60° C., 0.1×SSC, 0.1% SDS at 68° C. in another example.

As the probe, a part of the complementary sequences of the genes can also be used. Such a probe can be produced by PCR using oligonucleotides prepared on the basis of a known gene sequence as primers, and a DNA fragment including a nucleotide sequence of the gene as the template. When a DNA fragment having a length of about 300 by is used as the probe, the washing conditions after hybridization under the aforementioned conditions can be exemplified by 2×SSC, 0.1% SDS at 50° C.

The expression “to be modified so that an enzymatic activity of the meso-DAP synthesis pathway decreases” means to be modified so that the activity of an enzyme belonging to the meso-DAP synthesis pathway (also referred to as “DapDCEF pathway”), specifically, at least one of the four enzymes, DapD, DapC, DapE, and DapF, is completely eliminated or decreased as compared to that of a non-modified strain of Escherichia coli, for example, a wild-type strain.

The enzyme with decreased activity can be any of DapD, DapC, DapE, and DapF, and can include two or more of these. The activity of an enzyme functioning in the upstream region of the DapDCEF pathway can be decreased, and at least DapD can be modified to decrease the activity, in another example.

Decrease of the enzymatic activity of the DapDCEF pathway can mean that, for example, each enzymatic activity in the DapDCEF pathway can be decreased to 50% or less, 30% or less in another example, 10% or less in another example, per cell, as compared to that of a non-modified strain, for example, a wild-type strain.

Examples of strains that can be used for comparison include Escherichia coli W3110 (ATCC 27325), Escherichia coli MG1655 (ATCC 47076), and so forth, both of which are derived from the prototype wild-type strain.

A modification that results in a decrease of enzymatic activity in the DapDCEF pathway can include, specifically, deleting a part of, or the entire gene, on a chromosome coding for an enzyme of the DapDCEF pathway, specifically, the coding region of the dapD, dapC, dapE, or dapF gene, or modifying an expression control sequence such as promoter and Shine-Dalgarno (SD) sequence. Furthermore, expression of a gene can also be decreased by modifying a non-translation region, other than expression control sequence. Furthermore, the entire gene including the sequences on both sides of the gene on a chromosome can be deleted. Furthermore, the modification can include introducing a mutation which causes an amino acid substitution (missense mutation), a stop codon (nonsense mutation), or a frame shift mutation which adds or deletes one or two nucleotides, into a coding region coding for an enzyme on a chromosome (Journal of Biological Chemistry, 272:8611-8617 (1997); Proceedings of the National Academy of Sciences, USA, 95 5511-5515 (1998); Journal of Biological Chemistry, 266, 20833-20839 (1991)).

The intracellular enzymatic activity can be decreased by deleting a part of, or the entire expression regulatory sequence of the gene on a chromosome, such as a promoter region. Also, intracellular enzymatic activity can be decreased by deleting a part of, or the entire coding region. A non-coding region can also be deleted, or other sequences can be inserted into any of these regions using homologous recombination. However, the modification can also be attained by a typical mutagenesis caused by X-ray or ultraviolet irradiation, or by the use of a mutagene such as N-methyl-N′-nitro-N-nitrosoguanidine, so long as the modification results in a decrease of an enzymatic activity of the DapDCEF pathway.

An expression control sequence can be modified by mutating one or more nucleotides, two or more nucleotides in another example, or three or more nucleotides in another example. When a deletion occurs in a coding region, the region to be deleted can be the N-terminal region, an internal region, or the C-terminal region, or even the entire coding region, so long as the function of the enzyme protein to be produced is decreased or deleted. The longer the region which is deleted, the greater the likelihood of inactivating the gene. Furthermore, the reading frames located upstream and downstream of the deleted region can be the same or different.

To inactivate a gene by inserting a sequence into the coding region of the gene, the sequence can be inserted into any part of the coding region of the gene. The longer the inserted sequence, the greater the likelihood of inactivating the gene. Reading frames located upstream and downstream of the insertion site can be the same or different. The sequence to be inserted is not particularly limited so long as the insertion decreases or deletes the function of the enzyme protein, and examples include, for example, a transposon carrying an antibiotic resistance gene or a gene useful for L-lysine production, and so forth.

A gene on a chromosome can be modified as described above by, for example, preparing a deletion-type version of the gene in which a partial sequence of the gene is deleted so that the deletion-type gene does not produce a functional protein. Then, a bacterium can be transformed with a DNA containing the deletion-type gene to cause homologous recombination between the deletion-type gene and the native gene on the chromosome, and thereby substitute the deletion-type gene for the gene on the chromosome. The protein encoded by the deletion-type gene has a conformation different from that of the wild-type enzyme protein, if it is even produced, and thus the function is decreased or deleted. Such gene disruption based on gene substitution utilizing homologous recombination has been already reported, and includes Red-driven integration (Datsenko, K. A, and Wanner, B. L., Proc. Natl. Acad. Sci. USA, 97:6640-6645 (2000)), Red driven integration in combination with an excise system derived from phage (Cho, E. H., Gumport, R. I., Gardner, J. F., J. Bacteriol., 184:5200-5203 (2002)), use of a plasmid containing a temperature-sensitive replication origin or a plasmid capable of conjugative transfer, use of a suicide vector which lacks a replication origin in a host (U.S. Pat. No. 6,303,383, Japanese Patent Laid-open No. 05-007491), and so forth.

A decrease in expression of a gene can be confirmed by comparing the amount of mRNA transcribed from the gene with that in a wild-type or non-modified strain. The expression can be confirmed by Northern hybridization, RT-PCR (Molecular Cloning, Cold Spring Harbor Laboratory Press, Cold Spring Harbor, USA, 2001)), and so forth.

A decrease of the amount of a protein encoded by a gene can be confirmed by Western blotting using antibodies (Molecular Cloning, Cold spring Harbor Laboratory Press, Cold Spring Harbor, USA, 2001).

In order to introduce a gene coding for DDH (ddh) into Escherichia coli, for example, Escherichia coli can be transformed with a ddh gene by using a vector such as a plasmid or a phage. Examples of such vectors include pUC19, pUC18, pBR322, pHSG299, pHSG298, pHSG399, pHSG398, RSF1010, pMW119, pMW118, pMW219, pMW218, and so forth. Although the native promoter of the DDH gene can be used, so long as the gene can be expressed in Escherichia coli, a promoter that efficiently functions in Escherichia coli can also be used. Examples of such promoters include lac, trp, trc, tac, PR and PL promoter of λ phage, tet, and so forth.

The ddh gene can also be incorporated into the chromosome of Escherichia coli by using transduction, a transposon (Berg, D. E. and Berg, C. M., Bio/Technol., 1, 417 (1983)), Mu phage (Japanese Patent Laid-open No. 2-109985), or homologous recombination (Experiments in Molecular Genetics, Cold Spring Harbor Lab. (1972)). Furthermore, the copy number of the ddh gene can be increased by transferring ddh genes incorporated into the chromosome.

Incorporation of the ddh gene can be confirmed by, for example, Southern hybridization. Furthermore, whether the Escherichia coli bacterium into which the ddh gene has been introduced has the DDH activity or not can be confirmed by, for example, measuring the DDH activity according to the method described in Haruo Misono, Fermentation and Industry, 45, 964 (1987). Moreover, DDH can also be detected by Western blotting using an antibody.

<2> Method for Producing L-Lysine

The method for producing L-lysine can include the steps of culturing the bacterium in accordance with the presently disclosed subject matter in a medium to produce and accumulate L-lysine in the medium or cells, and collecting L-lysine from the medium or cells.

Media conventionally used in the production of L-lysine by fermentation using microorganisms can be used. That is, conventional media containing a carbon source, a nitrogen source, inorganic ions, and optionally other organic components as required can be used. As the carbon source, saccharides such as glucose, sucrose, lactose, galactose, fructose, and starch hydrolysate; alcohols such as glycerol and sorbitol; and organic acids such as fumaric acid, citric acid and succinic acid can be used. As the nitrogen source, inorganic ammonium salts such as ammonium sulfate, ammonium chloride, and ammonium phosphate, organic nitrogen such as soybean hydrolysate, ammonia gas, aqueous ammonia, and so forth can be used. As for organic trace nutrient sources, the medium can contain required substances such as vitamin B₁ and L-homoserine, yeast extract, or the like, in appropriate amounts. Other than the above, potassium phosphate, magnesium sulfate, iron ions, manganese ions and so forth are added in small amounts, as required. In addition, the medium can be either natural or synthetic, so long as the medium contains a carbon source, a nitrogen source, and inorganic ions, and other organic trace components as required.

Glycerol can be used as a carbon source. Although glycerol can be reagent-grade glycerol, industrially produced glycerol containing impurities can also be used. For example, glycerol industrially produced via esterification during biodiesel fuel production can be used (Mu Y, et al, Biotechnol Lett., 28, 1755-91759 (2006); Haas M. J., et al., Bioresour. Technol., 97, 4, 671-8678 (2006)).

Glycerol contained in the medium can be the sole carbon source, or other carbon sources can be added in addition to glycerol. Other possible carbon sources can include saccharides such as glucose, fructose, sucrose, lactose, galactose, blackstrap molasses, starch hydrolysate, sugar solution obtained by hydrolysis of biomass, alcohols such as ethanol, and organic acids such as fumaric acid, citric acid, and succinic acid. When a mixed medium is used, glycerol can be present in the medium in an amount of 50% or more, 60% or more in another example, 70% or more in another example, 80% or more in another example, 90% or more in another example, based on the total carbon source present in the medium.

The culture can be performed for 1 to 7 days under aerobic conditions. The culture temperature can be 24 to 37° C., and the pH during the culture can be between 5 to 9. To adjust the pH, inorganic or organic acidic, or alkaline substances, ammonia gas, and so forth can be used. To collect L-lysine from the fermentation medium, a combination of known methods can be used, such as by using an ion exchange resin or precipitation. When L-lysine accumulates in the cells, supersonic waves, for example, or the like, can be used to disrupt the cells, and L-lysine can be collected by using an ion exchange resin, or the like, from the supernatant remaining once the cells and cellular debris have been removed by centrifugation.

The production can also be performed by a method in which fermentation is performed by controlling the pH of the medium during culture to 6.5 to 9.0, controlling the pH of the medium at the end of the culture to 7.2 to 9.0, and controlling the pressure in the fermentation tank during fermentation so that it is positive. Alternatively, carbon dioxide or a mixed gas containing carbon dioxide can be added to the medium so that bicarbonate ions and/or carbonate ions accumulate in the culture medium in an amount of at least 2 g/L during the culture. These bicarbonate ions and/or carbonate ions can serve as counter ions of cations, mainly basic amino acid, and lysine is then collected (refer to Japanese Patent Laid-open No. 2002-065287, U.S. Published Patent Application No. 2002/025564).

### EXAMPLES

Hereinafter, the present invention will be still more specifically explained with reference to the following non-limiting examples.

### Example 1

### Construction of dapD-Disrupted L-Lysine-Producing Bacterium

1-1> Construction of dapD Gene-Disrupted Strain

First, a dapD-disrupted strain was constructed using the Escherichia coli wild-type strain, the MG1655 strain.

Using the pMW118 (λattL-Km^{r}-λattR) plasmid (International Patent Publication WO2006/093322) as the template, and synthetic oligonucleotide primers of SEQ ID NOS: 19 and 20, which have sequences corresponding to both ends of the attachment sites of λ phage, attL and attR, at the 3′ ends, and sequences corresponding to parts of the dapD gene as the target gene at the 5′ ends, PCR was performed to construct the MG1655ΔdapD::att-Km strain according to the λ-red method described in U.S. Patent Published Application No. 2006/0160191 and WO2005/010175. A Km-resistant recombinant was obtained according to the λ-red method by culturing the bacterium at 37° C. on an L-agar medium containing Km (kanamycin, 50 mg/L) as a plate culture and selecting a Km-resistant recombinant.

<1-2> Transduction of L-Lysine-Producing Bacterium WC196LC/pCABD2 Strain with ΔdapD::att-kan

P1 lysate was obtained from the MG1655ΔdapD::att-Km strain obtained in <1-1> in a conventional manner, and the L-lysine-producing bacterial strain WC196ΔcadAΔldcC/pCABD2 constructed by the method described in U.S. Patent Published Application No. 2006/0160191 was used as a host to construct the WC196ΔcadAΔldcCΔdapD::att-Km/pCABD2 strain by the P1 transduction method. The WC196ΔcadAΔldc strain was obtained from the Escherichia coli WC196 strain by disrupting the lysine decarboxylase genes, cadA and ldc, by using the Red-driven integration method (Datsenko K. A., Wanner, B. L., 2000, Proc. Natl. Acad. Sci. USA, 97, 6640-6645) and the excision system derived from λ-phage (Cho, E. H., Gumport, R. I., Gardner, J. F., J. Bacteriol., 184:5200-5203 (2002)) in combination (refer to WO2005/010175). The WC196ΔcadAΔldcC/pCABD2 strain was obtained by introducing pCABD2.

The objective transduced strain was obtained by performing a plate culture at 37° C. on an L-agar medium containing Km (kanamycin, 50 mg/L) and Sm (streptomycin, 20 mg/L), and selecting a Km-resistant and Sm-resistant recombinant strain.

Furthermore, these strains were each cultured at 37° C. in an L medium containing 20 mg/L of streptomycin until final OD600 reached about 0.6, then the culture was added with an equal volume of 40% glycerol solution, and the mixture was stirred, divided into appropriate volumes, and stored at −80° C. These mixtures are called glycerol stocks.

### Example 2

### Evaluation of L-Lysine Producing Ability of dapD-Disrupted L-Lysine-Producing Bacterium

The glycerol stocks of the strains obtained in Example 1 were thawed, and uniformly applied to an L-plate containing 20 mg/L of streptomycin in a volume of 100 μL each, and culture was performed at 37° C. for 24 hours. About ⅛ of the cells were scraped from one plate and suspended in 0.5 mL of physiological saline, and the turbidity of the suspension was measured at 600 nm using a spectrophotometer (U-2000, Hitachi). The suspension containing the obtained cells was inoculated into 20 mL of a fermentation medium (MS medium, composition is shown below) containing 20 mg/L of streptomycin in a 500-mL Sakaguchi flask in such a volume that turbidity of the mixture became 0.15 at 600 nm, and the culture was performed at 37° C. and 114 rpm for 24 hours on a reciprocally shaking culture machine. After the culture, the amounts of accumulated L-lysine and remaining glucose in the medium were measured using a Biotec Analyzer AS210 (SAKURA SEIKI). The amount of glycerol that also accumulated in the medium was measured using Biotec Analyzer BF-5 (Oji Scientific Instruments).

Composition of fermentation medium (g/L):

| | |
|---|---|
| Glycerol or glucose | 40 |
| (NH₄)₂SO₄ | 24 |
| KH₂PO₄ | 11.0 |
| MgSO₄•7H₂O | 1.0 |
| FeSO₄•7H₂O | 0.01 |
| MnSO₄•5H₂O | 0.01 |
| Yeast Extract | 2.0 |

The medium was adjusted to pH 7.0 with KOH, autoclaved at 115° C. for 10 minutes (provided that glycerol or glucose and MgSO₄.7H₂O were separately sterilized), and then 30 g/L of CaCO₃ was added (Japanese Pharmacopoeia) (subjected to hot air sterilization at 180° C. for 2 hours). Streptomycin was added in an amount of 20 mg/L.

The results are shown in Table 1 (OD indicates the cell amount indicated in terms of absorbance at 660 nm measured for culture diluted 26 times, Lys (g/L) means the amount of L-lysine that accumulated in the flask, Glucose (g/L) and Glycerol (g/L) mean the amounts of glucose and glycerol that remained in the medium, respectively, and Yield (%) means L-lysine yield based on the substrate). As seen from the results shown in Table 1, the WC196ΔcadAΔldcCΔdapD::att-Km/pCABD2 strain produced L-lysine in a larger amount as compared to that obtained with the WC196ΔcadAΔldcC/pCABD2 strain in which dapD gene was not disrupted.

TABLE 1

| | | | | |
|---|---|---|---|---|
| | O.D. | Lys | Glucose | Yield |
| Strain | (x26) | (g/L) | (g/L) | (%) |

MS glucose medium

| | | | | |
|---|---|---|---|---|
| WC196ΔcadAΔldcC/pCABD2 strain | 0.361 | 8.28 | 18.67 | 38.82 |
| WC196ΔcadAΔldcCΔdapD::att- | 0.377 | 8.75 | 18.13 | 40.01 |
| Km/pCABD2 strain | | | | |

MS glycerol medium

| | | | | |
|---|---|---|---|---|
| WC196ΔcadAΔldcC/pCABD2 strain | 0.248 | 2.79 | 28.64 | 24.57 |
| WC196ΔcadAΔldcCΔdapD::att- | 0.320 | 5.10 | 23.69 | 31.12 |
| Km/pCABD2 strain | | | | |

Explanation of Sequence Listing

SEQ ID NO: 1: Nucleotide sequence of E. coli dapD

SEQ ID NO: 2: Amino acid sequence of E. coli DapD

SEQ ID NO: 3: Nucleotide sequence of E. coli dapC

SEQ ID NO: 4: Amino acid sequence of E. coli DapC

SEQ ID NO: 5: Nucleotide sequence of E. coli dapE

SEQ ID NO: 6: Amino acid sequence of E. coli DapE

SEQ ID NO: 7: Nucleotide sequence of E. coli dapF

SEQ ID NO: 8: Amino acid sequence of E. coli DapF

SEQ ID NO: 9: Nucleotide sequence of ddh gene of C. glutamicum

SEQ ID NO: 10: Amino acid sequence of DDH of C. glutamicum

SEQ ID NO: 11: Nucleotide sequence of ddh gene of B. lactofermentum

SEQ ID NO: 12: Amino acid sequence of DDH of B. lactofermentum

SEQ ID NO: 13: Nucleotide sequence of ddh gene of C. efficiens

SEQ ID NO: 14: Amino acid sequence of DDH of C. efficiens

SEQ ID NO: 15: Nucleotide sequence of ddh gene of H. arsenicoxydans

SEQ ID NO: 16: Amino acid sequence of DDH of H. arsenicoxydans

SEQ ID NO: 17: Nucleotide sequence of ddh gene of B. thetaiotaomicron

SEQ ID NO: 18: Amino acid sequence of DDH of B. thetaiotaomicron

SEQ ID NO: 19: Primer for deletion of dapD gene

SEQ ID NO: 20: Primer for deletion of dapD gene

SEQ ID NO: 21: Nucleotide sequence of E. coli lysC

SEQ ID NO: 22: Amino acid sequence of E. coli LysC

SEQ ID NO: 23: Nucleotide sequence of E. coli dapA

SEQ ID NO: 24: Amino acid sequence of E. coli DapA

SEQ ID NO: 25: Nucleotide sequence of E. coli dapB

SEQ ID NO: 26: Amino acid sequence of E. coli DapB

### INDUSTRIAL APPLICABILITY

According to the present invention, in L-lysine production by fermentation using Escherichia coli, the production amount and/or fermentation yield of L-lysine can be improved. Moreover, the present invention can be used for the breeding of L-lysine-producing bacterium of Escherichia coli.

While the invention has been described in detail with reference to exemplary embodiments thereof, it will be apparent to one skilled in the art that various changes can be made, and equivalents employed, without departing from the scope of the invention. Each of the aforementioned documents is incorporated by reference herein in its entirety.

We claim:

1. An Escherichia coli bacterium which is able to produce L-lysine, wherein said bacterium has been modified to decrease an activity or activities of one or more enzymes of the meso-α,ε-diaminopimelic acid synthesis pathway, and wherein a gene coding for diaminopimelate dehydrogenase has been introduced into said bacterium.

2. The bacterium according to claim 1, wherein the enzymes of the meso-α,ε-diaminopimelic acid synthesis pathway are selected from the group consisting of 2,3,4,5-tetrahydropyridine-2,6-dicarboxylate N-succinyltransferase, succinyldiaminopimelate transaminase, succinyldiaminopimelate desuccinylase, and diaminopimelate epimerase.

3. The bacterium according to claim 2, wherein the 2,3,4,5-tetrahydropyridine-2,6-dicarboxylate N-succinyltransferase, succinyldiaminopimelate transaminase, succinyldiaminopimelate desuccinylase, and diaminopimelate epimerase are encoded by the dapD gene, dapC gene, dapE gene, and dapF gene, respectively.

4. The bacterium according to claim 3, wherein the activity or activities are decreased by a method selected from the group consisting of
1) decreasing expression of the gene or genes,
2) disrupting the gene or genes, and
3) combinations thereof.

5. The bacterium according to claim 2, which has been modified to decrease at least the activity of 2,3,4,5-tetrahydropyridine-2,6-dicarboxylate N-succinyltransferase.

6. The bacterium according to claim 1, wherein the gene coding for diaminopimelate dehydrogenase is the ddh gene of a coryneform bacterium.

7. The bacterium according to claim 2, wherein the 2,3,4,5-tetrahydropyridine-2,6-dicarboxylate N-succinyltransferase is selected from the group consisting of:
(a) the protein comprising the amino acid sequence of SEQ ID NO: 2, and
(b) the protein comprising the amino acid sequence of SEQ ID NO: 2, but wherein one or several amino acid residues are substituted, deleted, inserted or added, and the protein has 2,3,4,5-tetrahydropyridine-2,6-dicarboxylate N-succinyltransferase activity.

8. The bacterium according to claim 2, wherein the succinyldiaminopimelate transaminase is selected from the group consisting of:
(a) the protein comprising the amino acid sequence of SEQ ID NO: 4, and
(b) the protein comprising the amino acid sequence of SEQ ID NO: 4, but wherein one or several amino acid residues are substituted, deleted, inserted or added, and the protein has succinyldiaminopimelate transaminase activity.

9. The bacterium according to claim 2, wherein the succinyldiaminopimelate desuccinylase is selected from the group consisting of:
(a) the protein comprising the amino acid sequence of SEQ ID NO: 6, and
(b) the protein comprising the amino acid sequence of SEQ ID NO: 6, but wherein one or several amino acid residues are substituted, deleted, inserted or added, and the protein has succinyldiaminopimelate desuccinylase activity.

10. The bacterium according to claim 2, wherein the diaminopimelate epimerase is selected from the group consisting of:
(a) the protein comprising the amino acid sequence of SEQ ID NO: 8, and
(b) the protein comprising the amino acid sequence of SEQ ID NO: 8, but wherein one or several amino acid residues are substituted, deleted, inserted or added, and the protein has diaminopimelate epimerase activity.

11. The bacterium according to claim 3, wherein the dapD gene is selected from the group consisting of:
(a) the DNA comprising the nucleotide sequence of SEQ ID NO: 1, and
(b) a DNA which is able to hybridize with the nucleotide sequence of SEQ ID NO: 1, or a probe which can be prepared from the nucleotide sequence, under stringent conditions, and wherein said DNA encodes a protein having 2,3,4,5-tetrahydropyridine-2,6-dicarboxylate N-succinyltransferase activity.

12. The bacterium according to claim 3, wherein the dapC gene is selected from the group consisting of:
(a) the DNA comprising the nucleotide sequence of SEQ ID NO: 3, and
(b) a DNA which is able to hybridize with the nucleotide sequence of SEQ ID NO: 3, or a probe which can be prepared from the nucleotide sequence, under stringent conditions, and wherein said DNA encodes a protein having succinyldiaminopimelate transaminase activity.

13. The bacterium according to claim 3, wherein the dapE gene is selected from the group consisting of:
(a) the DNA comprising the nucleotide sequence of SEQ ID NO: 5, and
(b) a DNA which is able to hybridize with the nucleotide sequence of SEQ ID NO: 5, or a probe which can be prepared from the nucleotide sequence, under stringent conditions, and wherein said DNA encodes a protein having succinyldiaminopimelate desuccinylase activity.

14. The bacterium according to claim 3, wherein the dapF gene is selected from the group consisting of:
(a) the DNA comprising the nucleotide sequence of SEQ ID NO: 7, and
(b) a DNA which is able to hybridize with the nucleotide sequence of SEQ ID NO: 7, or a probe which can be prepared from the nucleotide sequence, under stringent conditions, and wherein said DNA encodes a protein having diaminopimelate epimerase activity.

15. The bacterium according to claim 1, wherein the diaminopimelate dehydrogenase is selected from the group consisting of:
(a) the protein comprising the amino acid sequence selected from the group consisting of SEQ ID NO: 10, 12, 14, 16, and 18, and
(b) the protein comprising the amino acid sequence selected from the group consisting of SEQ ID NO: 10, 12, 14, 16, and 18, but wherein one or several amino acid residues are substituted, deleted, inserted or added in said amino acid sequence, and the protein has diaminopimelate dehydrogenase activity.

16. The bacterium according to claim 6, wherein the ddh gene is selected from the group consisting of:
(a) the DNA comprising the nucleotide sequence selected from the group consisting of SEQ ID NO: 9, 11, 13, 15, and 17, and
(j) a DNA which is able to hybridize with the nucleotide sequence selected from the group consisting of SEQ ID NO: 9, 11, 13, 15, and 17, or a probe which can be prepared from the nucleotide sequence, under stringent conditions, and wherein said DNA encodes a protein having diaminopimelate dehydrogenase activity.

17. The bacterium according to claim 1, which further has:
a) a dihydrodipicolinate synthase which is desensitized to feedback inhibition by L-lysine,
b_an aspartokinase which is desensitized to feedback inhibition by L-lysine, and
c) enhanced activity of dihydrodipicolinate reductase.

18. A method for producing L-lysine, which comprises culturing the bacterium according to claim 1 in a medium and collecting L-lysine from the medium.


SEQUENCE LISTING (26 sequences)

SEQ ID NO: 1 - DNA, 825 bp, Escherichia coli
  CDS (1)..(825)
atgcagcagttacagaacattattgaaaccgcttttgaacgccgtgccgagatcacgccagccaatgcagacaccgttacccgcgaagcggtaaatcaggtgatcgccctgctggattccggcgcactgcgtgtagcggaaaaaattgacggtcagtgggtgacgcatcagtggttgaaaaaagcggtgctgctctctttccgtattaatgataatcaggtgatcgaaggggcagaaagccgctacttcgacaaagtgccgatgaaattcgccgactacgacgaagcacgtttccagaaagaaggcttccgcgttgtgccaccagcggcggtacgtcagggtgcgtttattgcccgtaacaccgtgctgatgccgtcttacgtcaacatcggcgcatatgttgatgaaggcaccatggttgatacctgggcgaccgtcggttcttgtgcgcagattggtaaaaacgtccacctttccggtggcgtgggcatcggcggcgtgctggaaccgctgcaggctaacccaaccatcattgaagataattgcttcatcggcgcgcgctctgaagtggttgaaggggtgattgtcgaagaaggttccgtcatttccatgggcgtatacattggtcagagcacccgtatttacgaccgtgaaaccggcgaaatccactacggtcgcgttccggcggggtctgtggttgtttcaggtaatctgccgtcaaaagatggcaaatacagcctctactgtgcggttatcgttaagaaagttgacgcgaaaactcgcggcaaagtcggcattaacgaactgctgcgtaccatcgactaa

SEQ ID NO: 2 - protein, 274 aa, Escherichia coli
MQQLQNIIETAFERRAEITPANADTVTREAVNQVIALLDSGALRVAEKIDGQWVTHQWLKKAVLLSFRINDNQVIEGAESRYFDKVPMKFADYDEARFQKEGFRVVPPAAVRQGAFIARNTVLMPSYVNIGAYVDEGTMVDTWATVGSCAQIGKNVHLSGGVGIGGVLEPLQANPTIIEDNCFIGARSEVVEGVIVEEGSVISMGVYIGQSTRIYDRETGEIHYGRVPAGSVVVSGNLPSKDGKYSLYCAVIVKKVDAKTRGKVGINELLRTID

SEQ ID NO: 3 - DNA, 1221 bp, Escherichia coli
  CDS (1)..(1221)
atggcaattgaacaaacagcaattacacgcgcgactttcgatgaagtgatcctgccgatttatgctccggcagagtttattccggtaaaaggtcagggcagccgaatctgggatcagcaaggcaaggagtatgtcgatttcgcgggtggcattgcagttacggcgttgggccattgccatcctgcgctggtgaacgcgttaaaaacccagggcgaaactctgtggcatatcagtaacgttttcaccaatgaaccggcgctgcgtcttgggcgtaaactgattgaggcaacgtttgccgaacgcgtggtgtttatgaactccggcacggaagctaacgaaaccgcctttaaactggcacgccattacgcctgtgtgcgtcatagcccgttcaaaaccaaaattattgccttccataacgcttttcatggtcgctcgctgtttaccgtttcggtgggtgggcagccaaaatattccgacggctttgggccgaaaccggcagacatcatccacgttccctttaacgatctccatgcagtgaaagcggtgatggatgatcacacctgtgcggtggtggttgagccgatccagggcgagggcggtgtgacggcagcgacgccagagtttttgcagggcttgcgcgagctgtgcgatcaacatcaggcattattggtgtttgatgaagtgcagtgcgggatggggcggaccggcgatttgtttgcttacatgcactacggcgttacgccggatattctgacctctgcgaaagcgttaggcggcggcttcccgattagcgccatgctgaccacggcggaaattgcttctgcgtttcatcctggttctcacggttccacctacggcggtaatcctctggcctgtgcagtagcgggggcggcgtttgatatcatcaatacccctgaagtgctggaaggcattcaggcgaaacgccagcgttttgttgaccatctgcagaagatcgatcagcagtacgatgtatttagcgatattcgcggtatggggctgttgattggcgcagagctgaaaccacagtacaaaggtcgggcgcgtgatttcctgtatgcgggcgcagaggctggcgtaatggtgctgaatgccggaccggatgtgatgcgttttgcaccgtcgctggtggtggaagatgcggatatcgatgaagggatgcaacgtttcgcccacgcggtggcgaaggtggttggggcgtaa

SEQ ID NO: 4 - protein, 406 aa, Escherichia coli
MAIEQTAITRATFDEVILPIYAPAEFIPVKGQGSRIWDQQGKEYVDFAGGIAVTALGHCHPALVNALKTQGETLWHISNVFTNEPALRLGRKLIEATFAERVVFMNSGTEANETAFKLARHYACVRHSPFKTKIIAFHNAFHGRSLFTVSVGGQPKYSDGFGPKPADIIHVPFNDLHAVKAVMDDHTCAVVVEPIQGEGGVTAATPEFLQGLRELCDQHQALLVFDEVQCGMGRTGDLFAYMHYGVTPDILTSAKALGGGFPISAMLTTAEIASAFHPGSHGSTYGGNPLACAVAGAAFDIINTPEVLEGIQAKRQRFVDHLQKIDQQYDVFSDIRGMGLLIGAELKPQYKGRARDFLYAGAEAGVMVLNAGPDVMRFAPSLVVEDADIDEGMQRFAHAVAKVVGA

SEQ ID NO: 5 - DNA, 1128 bp, Escherichia coli
  CDS (1)..(1128)
atgtcgtgcccggttattgagctgacacaacagcttattcgccgcccttccctgagtcctgatgatgcaggatgccaggctttgttgattgaacgtttgcaggcgatcggttttaccgttgaacgcatggactttgccgatacgcagaatttttgggcatggcgtgggcagggtgaaacgttagcctttgccgggcataccgacgtggtgccgcctggcgacgccgatcgttggatcaatcccccgtttgaacccaccattcgtgacggcatgttattcgggcgcggtgcggcagatatgaaaggctcgctggcggcgatggtggtggcggcagaacgttttgtcgcacaacatcccaaccatacggggcgactggcatttctgatcacctctgatgaagaagccagtgcccacaacggtacggtaaaagtcgtcgaagcgttaatggcacgtaatgagcgtctcgattactgcctggttggcgaaccgtcgagtatcgaagtggtaggtgatgtggtgaaaaatggtcgtcgcggatcattaacctgcaaccttaccattcatggcgttcaggggcatgttgcctacccacatctggctgacaatccggtacatcgcgcagcacctttccttaatgaattagtggctattgagtgggatcagggcaatgaattcttcccggcgaccagtatgcagattgccaatattcaggcgggaacgggcagtaacaacgttattccgggtgaactgtttgtgcagtttaacttccgcttcagcaccgaactgactgatgagatgatcaaagcgcaggtgcttgccctgcttgaaaaacatcaactgcgctatacggtggattggtggctttccgggcagccatttttgaccgcgcgcggtaaactggtggatgcggtcgttaacgcggttgagcactataatgaaattaaaccgcagctactgaccacaggcggaacgtccgacgggcgctttattgcccgcatgggggcgcaggtggtggaactcgggccggtcaatgccactattcataaaattaatgaatgtgtgaacgctgccgacctgcagctacttgcccgtatgtatcaacgtatcatggaacagctcgtcgcctga

SEQ ID NO: 6 - protein, 375 aa, Escherichia coli
MSCPVIELTQQLIRRPSLSPDDAGCQALLIERLQAIGFTVERMDFADTQNFWAWRGQGETLAFAGHTDVVPPGDADRWINPPFEPTIRDGMLFGRGAADMKGSLAAMVVAAERFVAQHPNHTGRLAFLITSDEEASAHNGTVKVVEALMARNERLDYCLVGEPSSIEVVGDVVKNGRRGSLTCNLTIHGVQGHVAYPHLADNPVHRAAPFLNELVAIEWDQGNEFFPATSMQIANIQAGTGSNNVIPGELFVQFNFRFSTELTDEMIKAQVLALLEKHQLRYTVDWWLSGQPFLTARGKLVDAVVNAVEHYNEIKPQLLTTGGTSDGRFIARMGAQVVELGPVNATIHKINECVNAADLQLLARMYQRIMEQLVA

SEQ ID NO: 7 - DNA, 825 bp, Escherichia coli
  CDS (1)..(825)
atgcagttctcgaaaatgcatggccttggcaacgattttatggtcgtcgacgcggtaacgcagaatgtctttttttcaccggagctgattcgtcgcctggctgatcggcacctgggggtagggtttgaccaactgctggtggttgagccgccgtatgatcctgaactggattttcactatcgcattttcaatgctgatggcagtgaagtggcgcagtgcggcaacggtgcgcgctgctttgcccgttttgtgcgtctgaaaggactgaccaataagcgtgatatccgcgtcagcaccgccaacgggcggatggttctgaccgtcaccgatgatgatctggtccgcgtaaatatgggcgaacccaacttcgaaccttccgccgtgccgtttcgcgctaacaaagcggaaaagacctatattatgcgcgccgccgagcagacaatcttatgcggcgtggtgtcgatgggaaatccgcattgcgtgattcaggtcgatgatgtcgataccgcggcggtagaaacgcttggtcctgttctggaaagccacgagcgttttccggagcgcgccaatatcggttttatgcaagtggttaagcgcgagcatattcgtttacgcgtttatgagcgtggggcaggagaaacccaggcctgcggcagcggcgcgtgtgcggcggttgcagtagggattcagcaaggtttgctggccgaagaagtacgcgtggaactccccggcggtcgtcttgatatcgcctggaaaggtccgggtcacccgttatatatgactggcccggcggtacatgtctacgacggatttattcatctatga

SEQ ID NO: 8 - protein, 274 aa, Escherichia coli
MQFSKMHGLGNDFMVVDAVTQNVFFSPELIRRLADRHLGVGFDQLLVVEPPYDPELDFHYRIFNADGSEVAQCGNGARCFARFVRLKGLTNKRDIRVSTANGRMVLTVTDDDLVRVNMGEPNFEPSAVPFRANKAEKTYIMRAAEQTILCGVVSMGNPHCVIQVDDVDTAAVETLGPVLESHERFPERANIGFMQVVKREHIRLRVYERGAGETQACGSGACAAVAVGIQQGLLAEEVRVELPGGRLDIAWKGPGHPLYMTGPAVHVYDGFIHL

SEQ ID NO: 9 - DNA, 963 bp, Corynebacterium glutamicum
  CDS (1)..(963)
atgaccaacatccgcgtagctatcgtgggctacggaaacctgggacgcagcgtcgaaaagcttattgccaagcagcccgacatggaccttgtaggaatcttctcgcgccgggccaccctcgacacaaagacgccagtctttgatgtcgccgacgtggacaagcacgccgacgacgtggacgtgctgttcctgtgcatgggctccgccaccgacatccctgagcaggcaccaaagttcgcgcagttcgcctgcaccgtagacacctacgacaaccaccgcgacatcccacgccaccgccaggtcatgaacgaagccgccaccgcagccggcaacgttgcactggtctctaccggctgggatccaggaatgttctccatcaaccgcgtctacgcagcggcagtcttagccgagcaccagcagcacaccttctggggcccaggtttgtcacagggccactccgatgctttgcgacgcatccctggcgttcaaaaggcagtccagtacaccctcccatccgaagacgccctggaaaaggcccgccgcggcgaagccggcgaccttaccggaaagcaaacccacaagcgccaatgcttcgtggttgccgacgcggccgatcacgagcgcatcgaaaacgacatccgcaccatgcctgattacttcgttggctacgaagtcgaagtcaacttcatcgacgaagcaaccttcgactccgagcacaccggcatgccacacggtggccacgtgattaccaccggcgacaccggtggcttcaaccacaccgtggaatacatcctcaagctggaccgaaacccagatttcaccgcttcctcacagatcgctttcggtcgcgcagctcaccgcatgaagcagcagggccaaagcggagctttcaccgtcctcgaagttgctccatacctgctctccccagagaacttggacgatctgatcgcacgcgacgtctaa

SEQ ID NO: 10 - protein, 320 aa, Corynebacterium glutamicum
MTNIRVAIVGYGNLGRSVEKLIAKQPDMDLVGIFSRRATLDTKTPVFDVADVDKHADDVDVLFLCMGSATDIPEQAPKFAQFACTVDTYDNHRDIPRHRQVMNEAATAAGNVALVSTGWDPGMFSINRVYAAAVLAEHQQHTFWGPGLSQGHSDALRRIPGVQKAVQYTLPSEDALEKARRGEAGDLTGKQTHKRQCFVVADAADHERIENDIRTMPDYFVGYEVEVNFIDEATFDSEHTGMPHGGHVITTGDTGGFNHTVEYILKLDRNPDFTASSQIAFGRAAHRMKQQGQSGAFTVLEVAPYLLSPENLDDLIARDV

SEQ ID NO: 11 - DNA, 963 bp, Brevibacterium lactofermentum
  CDS (1)..(963)
atgaccaacatccgcgtagctatcgtaggctacggaaacctgggacgcagcgtcgaaaagcttattgccaagcagcccgacatggaccttgtaggaatcttctcgcgccgggccaccctcgacacaaagacgccagtctttgatgtcgccgacgtggacaagcacgccgacgacgtggacgtgctgttcctgtgcatgggctccgccaccgacatccctgagcaggcaccaaagttcgcgcagttcgcctgcaccgtagacacctacgacaaccaccgcgacatcccacgccaccgccaggtcatgaacgaagccgccaccgcagccggcaacgttgcactggtctctaccggctgggatccaggaatgttctccatcaaccgcgtctacgcagcggcagtcttagccgagcaccagcagcacaccttctggggcccaggtttgtcacagggccactccgatgctttgcgacgcatccctggcgttcaaaaggccgtccagtacaccctcccatccgaagaagccctggaaaaggcccgccgtggcgaagccggcgacctcaccggaaagcaaacccacaagcgccaatgcttcgtggttgccgacgcggccgaccacgagcgcatcgaaaacgacatccgcaccatgcctgattacttcgttggctacgaagtcgaagtcaacttcatcgacgaagcaaccttcgacgccgagcacaccggcatgccacacggcggacacgtgatcaccaccggcgacaccggtggcttcaaccacaccgtggaatacatcctgaagctggaccgaaacccagatttcaccgcttcttcacagatcgctttcggccgcgcagctcaccgcatgaagcagcagggccaaagcggtgccttcaccgtcctcgaagttgctccatacttgctctccccggagaacttggatgatctgatcgcacgcgacgtctaa

SEQ ID NO: 12 - protein, 320 aa, Brevibacterium lactofermentum
MTNIRVAIVGYGNLGRSVEKLIAKQPDMDLVGIFSRRATLDTKTPVFDVADVDKHADDVDVLFLCMGSATDIPEQAPKFAQFACTVDTYDNHRDIPRHRQVMNEAATAAGNVALVSTGWDPGMFSINRVYAAAVLAEHQQHTFWGPGLSQGHSDALRRIPGVQKAVQYTLPSEEALEKARRGEAGDLTGKQTHKRQCFVVADAADHERIENDIRTMPDYFVGYEVEVNFIDEATFDAEHTGMPHGGHVITTGDTGGFNHTVEYILKLDRNPDFTASSQIAFGRAAHRMKQQGQSGAFTVLEVAPYLLSPENLDDLIARDV

SEQ ID NO: 13 - DNA, 963 bp, Corynebacterium efficiens
  CDS (1)..(963)
atgtcgaagatccgcgcagcaatcgttggttatggaaatctggggaagagcgtcgagaagctcatcgtccagcaaccggacatggaactggtggggatcttctcccgccgcgacaccctggacaccgacacccccgtgttcaacgtcgccgagacggagaagcacaccggcgatgttgatctcctcttcctctgcatgggttccgccactgacatcccggagcaggccccgggttttgcggcattcgcctgcaccgtggacacctatgacaaccaccgggacatcccgcgtcaccgtcaggtgatggatgaggccgcccgtgccgccggcaatgtctctgttgtcgccaccggttgggatccggggatgttctccatcaaccgcgtgtacggcgcagccctgctcgccgatcaccagcagcacaccttctggggaccgggtctgtcccagggccactccgatgccttgcgacgcatcgacggcgtcgagaaggccgtccagtacaccctgccttccgaggatgccctggagaaggcacgccgcggtgaggctgaggggctgaccggtaaacagacccacaagcgtcagtgtttcgtggtcgccccggagtccgagcacgagcgcatcgagaatgagatccgcaccatggctgactacttcgtcggctatgaggtggaggtcaacttcatcgatgaggctaccttcgattccgagcacaccggaatgccccacggcggtcatgtgatcaccaccggtgacaccggcggtttccaccacactgtggagtacaccctgaagctggatcgcaaccctgacttcaccgcctcctcccagattgcgttcggacgtgctgcctaccgactgaaggaagcaggtcaggctggggcattcaccgttctggaggtcgctccctacctcctgtccccgacaccactcgatgacctgatcgcccgcgacgtctag

SEQ ID NO: 14 - protein, 320 aa, Corynebacterium efficiens
MSKIRAAIVGYGNLGKSVEKLIVQQPDMELVGIFSRRDTLDTDTPVFNVAETEKHTGDVDLLFLCMGSATDIPEQAPGFAAFACTVDTYDNHRDIPRHRQVMDEAARAAGNVSVVATGWDPGMFSINRVYGAALLADHQQHTFWGPGLSQGHSDALRRIDGVEKAVQYTLPSEDALEKARRGEAEGLTGKQTHKRQCFVVAPESEHERIENEIRTMADYFVGYEVEVNFIDEATFDSEHTGMPHGGHVITTGDTGGFHHTVEYTLKLDRNPDFTASSQIAFGRAAYRLKEAGQAGAFTVLEVAPYLLSPTPLDDLIARDV

SEQ ID NO: 15 - DNA, 987 bp, Herminiimonas arsenicoxydans
  CDS (1)..(987)
atggatgaaaaaatccgtattggggttgccggatacggtaacctcggtcgcggggtcgaaatggcgatcgcacgcaaccctgatatgcaactggtcggcgttttcagccggcgcgatcccgctagcatcgagcttttgacccaggctgtacccgtccataaattcgacgatatcgaacagtttcgcgaccagattgacgtgctcattctgtgcggcggatcaaagaacgatctgcccgaacaaggcccagcattggctaccttgttcaacacgattgacagctttgatacacacaataaaattccggaatatttcgccgcgatggacagtgcctcatgcgttggcgagcgcacatccatcatctcggtaggctgggatccgggcgtgttctcgctcaatcgtctgttcggcgaagccattttgccagaaggcgaaacttataccttctggggtaaaggtctaagtcaggggcactcggacgcaatacgccgcgtgccgggcgtcaaggccggtgtgcagtacacgatcccctcggctgaggcgatggaactggtacgcagtggcagccagccccaactgtcgacacgcgagaaacatactcgcgagtgtcatgttgtactcgaagccggtgccgatgccaaagcggtggaacatgccatcgtcagcatgccggactatttcgccgactatgacacgaccgtgcacttcatcagcgaggaagagttacgaagcaatcactctgccatgccacacggaggcttcgttatccgcagcggccagacaggagatggcagcaaacaggtaatagagtattcgctcaagctcggtagcaatcctgaatttacagccgcggtactagtcgcttacgcccgcgctgctttccgcctccacaaaaaaggagtgcacggagcgcacagcgtgctggatatcgctccgggccttctgtcacccaaaagtccggctcagttgcgcaaagagttgctctga

SEQ ID NO: 16 - protein, 328 aa, Herminiimonas arsenicoxydans
MDEKIRIGVAGYGNLGRGVEMAIARNPDMQLVGVFSRRDPASIELLTQAVPVHKFDDIEQFRDQIDVLILCGGSKNDLPEQGPALATLFNTIDSFDTHNKIPEYFAAMDSASCVGERTSIISVGWDPGVFSLNRLFGEAILPEGETYTFWGKGLSQGHSDAIRRVPGVKAGVQYTIPSAEAMELVRSGSQPQLSTREKHTRECHVVLEAGADAKAVEHAIVSMPDYFADYDTTVHFISEEELRSNHSAMPHGGFVIRSGQTGDGSKQVIEYSLKLGSNPEFTAAVLVAYARAAFRLHKKGVHGAHSVLDIAPGLLSPKSPAQLRKELL

SEQ ID NO: 17 - DNA, 900 bp, Bacteroides thetaiotaomicron
  CDS (1)..(900)
atgaaaaaagtaagagcagctattgtcggttacggcaatatcggacactatgtacttgaagcgctacaggcagcgcctgatttcgaaatagccggagtagttcgtcgtgcaggagcagagaacaagccggaagagttggcaaactatgcagtagtcaaagatatcaaagagctggaaggagtggaagtggccatcctctgcacaccgacccgcagcgttgagaaatacgcgaaagaatatttggcaatgggaatcaacacggtggacagcttcgacatccacacaggcatcgttgacctgcgccgcacgctggatgccaccgccaaagagcacaaagccgtatccatcatctccgcaggatgggatccgggaagcgactcgatcgtacgcaccatgctcgaagcaatcgctccgaaaggcatcacttacaccaacttcggtcccggcatgagtatgggacacaccgtagccgtaaaagcgatcgacggagtgaaagccgccctctccatgacgatccctactggaacgggaatccaccgccgcatggtatatatcgaactgaaagacggatacaagtttgaagaagtagccgcagccatcaaggcagacccttacttcgtgaacgacgagacacatgtaaaacttgtgcccagcgtagacgcactgctcgatatgggacacggtgtaaatctgactcgcaagggagtttccggtaaaacgcagaatcagctgttcgagttcaatatgcgcatcaacaatccggcactgaccgcacaggtacttgtgtgcgtggcacgcgcttccatgaagcagcaaccggggtgctacaccatggtagaagtccccgtcatcgacctccttccgggcgaccgcgaagagtggatcggacacctggtataa

SEQ ID NO: 18 - protein, 299 aa, Bacteroides thetaiotaomicron
MKKVRAAIVGYGNIGHYVLEALQAAPDFEIAGVVRRAGAENKPEELANYAVVKDIKELEGVEVAILCTPTRSVEKYAKEYLAMGINTVDSFDIHTGIVDLRRTLDATAKEHKAVSIISAGWDPGSDSIVRTMLEAIAPKGITYTNFGPGMSMGHTVAVKAIDGVKAALSMTIPTGTGIHRRMVYIELKDGYKFEEVAAAIKADPYFVNDETHVKLVPSVDALLDMGHGVNLTRKGVSGKTQNQLFEFNMRINNPALTAQVLVCVARASMKQQPGCYTMVEVPVIDLLPGDREEWIGHLV

SEQ ID NO: 19 - DNA, 69 bp, Artificial sequence
  primer
atgcagcagttacagaacattattgaaaccgcttttgaacgccgtgccgatctagacgctcaagttagt

SEQ ID NO: 20 - DNA, 70 bp, Artificial sequence
  primer
ttagtcgatggtacgcagcagttcgttaatgccgactttgccgcgagtttagatcttgaagcctgctttt

SEQ ID NO: 21 - DNA, 1350 bp, Escherichia coli
  CDS (1)..(1350)
atgtctgaaattgttgtctccaaatttggcggtaccagcgtagctgattttgacgccatgaaccgcagcgctgatattgtgctttctgatgccaacgtgcgtttagttgtcctctcggcttctgctggtatcactaatctgctggtcgctttagctgaaggactggaacctggcgagcgattcgaaaaactcgacgctatccgcaacatccagtttgccattctggaacgtctgcgttacccgaacgttatccgtgaagagattgaacgtctgctggagaacattactgttctggcagaagcggcggcgctggcaacgtctccggcgctgacagatgagctggtcagccacggcgagctgatgtcgaccctgctgtttgttgagatcctgcgcgaacgcgatgttcaggcacagtggtttgatgtacgtaaagtgatgcgtaccaacgaccgatttggtcgtgcagagccagatatagccgcgctggcggaactggccgcgctgcagctgctcccacgtctcaatgaaggcttagtgatcacccagggatttatcggtagcgaaaataaaggtcgtacaacgacgcttggccgtggaggcagcgattatacggcagccttgctggcggaggctttacacgcatctcgtgttgatatctggaccgacgtcccgggcatctacaccaccgatccacgcgtagtttccgcagcaaaacgcattgatgaaatcgcgtttgccgaagcggcagagatggcaacttttggtgcaaaagtactgcatccggcaacgttgctacccgcagtacgcagcgatatcccggtctttgtcggctccagcaaagacccacgcgcaggtggtacgctggtgtgcaataaaactgaaaatccgccgctgttccgcgctctggcgcttcgtcgcaatcagactctgctcactttgcacagcctgaatatgctgcattctcgcggtttcctcgcggaagttttcggcatcctcgcgcggcataatatttcggtagacttaatcaccacgtcagaagtgagcgtggcattaacccttgataccaccggttcaacctccactggcgatacgttgctgacgcaatctctgctgatggagctttccgcactgtgtcgggtggaggtggaagaaggtctggcgctggtcgcgttgattggcaatgacctgtcaaaagcctgcggcgttggcaaagaggtattcggcgtactggaaccgttcaacattcgcatgatttgttatggcgcatccagccataacctgtgcttcctggtgcccggcgaagatgccgagcaggtggtgcaaaaactgcatagtaatttgtttgagtaa

SEQ ID NO: 22 - protein, 449 aa, Escherichia coli
MSEIVVSKFGGTSVADFDAMNRSADIVLSDANVRLVVLSASAGITNLLVALAEGLEPGERFEKLDAIRNIQFAILERLRYPNVIREEIERLLENITVLAEAAALATSPALTDELVSHGELMSTLLFVEILRERDVQAQWFDVRKVMRTNDRFGRAEPDIAALAELAALQLLPRLNEGLVITQGFIGSENKGRTTTLGRGGSDYTAALLAEALHASRVDIWTDVPGIYTTDPRVVSAAKRIDEIAFAEAAEMATFGAKVLHPATLLPAVRSDIPVFVGSSKDPRAGGTLVCNKTENPPLFRALALRRNQTLLTLHSLNMLHSRGFLAEVFGILARHNISVDLITTSEVSVALTLDTTGSTSTGDTLLTQSLLMELSALCRVEVEEGLALVALIGNDLSKACGVGKEVFGVLEPFNIRMICYGASSHNLCFLVPGEDAEQVVQKLHSNLFE

SEQ ID NO: 23 - DNA, 879 bp, Escherichia coli
  CDS (1)..(879)
atgttcacgggaagtattgtcgcgattgttactccgatggatgaaaaaggtaatgtctgtcgggctagcttgaaaaaactgattgattatcatgtcgccagcggtacttcggcgatcgtttctgttggcaccactggcgagtccgctaccttaaatcatgacgaacatgctgatgtggtgatgatgacgctggatctggctgatgggcgcattccggtaattgccgggaccggcgctaacgctactgcggaagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaacgcgtgtaaaccagatcaaagagctggtttcagatgattttgttctgctgagcggcgatgatgcgagcgcgctggacttcatgcaattgggcggtcatggggttatttccgttacggctaacgtcgcagcgcgtgatatggcccagatgtgcaaactggcagcagaagggcattttgccgaggcacgcgttattaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggtcgtgagacggtcagagcggcgcttaagcatgccggtttgctgtaa

SEQ ID NO: 24 - protein, 292 aa, Escherichia coli
MFTGSIVAIVTPMDEKGNVCRASLKKLIDYHVASGTSAIVSVGTTGESATLNHDEHADVVMMTLDLADGRIPVIAGTGANATAEAISLTQRFNDSGIVGCLTVTPYYNRPSQEGLYQHFKAIAEHTDLPQILYNVPSRTGCDLLPETVGRLAKVKNIIGIKEATGNLTRVNQIKELVSDDFVLLSGDDASALDFMQLGGHGVISVTANVAARDMAQMCKLAAEGHFAEARVINQRLMPLHNKLFVEPNPIPVKWACKELGLVATDTLRLPMTPITDSGRETVRAALKHAGLL

SEQ ID NO: 25 - DNA, 822 bp, Escherichia coli
  CDS (1)..(822)
atgcatgatgcaaacatccgcgttgccatcgcgggagccggggggcgtatgggccgccagttgattcaggcggcgctggcattagagggcgtgcagttgggcgctgcgctggagcgtgaaggatcttctttactgggcagcgacgccggtgagctggccggagccgggaaaacaggcgttaccgtgcaaagcagcctcgatgcggtaaaagatgattttgatgtgtttatcgattttacccgtccggaaggtacgctgaaccatctcgctttttgtcgccagcatggcaaagggatggtgatcggcactacggggtttgacgaagccggtaaacaagcaattcgtgacgccgctgccgatattgcgattgtctttgctgccaattttagcgttggcgttaacgtcatgcttaagctgctggagaaagcagccaaagtgatgggtgactacaccgatatcgaaattattgaagcacatcatagacataaagttgatgcgccgtcaggcaccgcactggcaatgggagaggcgatcgcccacgcccttgataaagatctgaaagattgcgcggtctacagtcgtgaaggccacaccggtgaacgtgtgcctggcaccattggttttgccaccgtgcgtgcaggtgacatcgttggtgaacataccgcgatgtttgccgatattggcgagcgtctggagatcacccataaggcgtccagccgtatgacatttgctaacggcgcggtaagatcggctttgtggttgagtggtaaggaaagcggtctttttgatatgcgagatgtacttgatctcaataatttgtaa

SEQ ID NO: 26 - protein, 273 aa, Escherichia coli
MHDANIRVAIAGAGGRMGRQLIQAALALEGVQLGAALEREGSSLLGSDAGELAGAGKTGVTVQSSLDAVKDDFDVFIDFTRPEGTLNHLAFCRQHGKGMVIGTTGFDEAGKQAIRDAAADIAIVFAANFSVGVNVMLKLLEKAAKVMGDYTDIEIIEAHHRHKVDAPSGTALAMGEAIAHALDKDLKDCAVYSREGHTGERVPGTIGFATVRAGDIVGEHTAMFADIGERLEITHKASSRMTFANGAVRSALWLSGKESGLFDMRDVLDLNNL